(19) United States
(12) Patent Application Publication
(10) Pub. No.: US 2016/0235355 A1
(43) Pub. Date: Aug. 18, 2016

(54) METHOD FOR EVALUATING THE SEVERITY OF ATROPHIC ACNE SCARS ON A PATIENT AND DEVICE USED TO EVALUATE THE DISTRIBUTION OF ACNE SCARS

(71) Applicant: GALDERMA RESEARCH & DEVELOPMENT, Biot (FR)

(72) Inventor: Laurent PETIT, Peymeinade (FR)

(73) Assignee: GALDERMA RESEARCH & DEVELOPMENT, Biot (FR)

(21) Appl. No.: 14/780,360

(22) PCT Filed: Mar. 28, 2014
(86) PCT No.: PCT/EP2014/056382

§ 371 Date: May 2, 2016

(30) Foreign Application Priority Data
Mar. 29, 2013 (EP) 13305419.7

(51) Int. Cl. A61B 5/00

(52) U.S. Cl. CPC A61B 5/444; A61B 5/6814; A61B 2562/164

(57) ABSTRACT

A method is described for evaluating the severity of atrophic acne scars in a patient. Also described, is a device for implementing this method. The device can be in the form of a transparent support made of flexible plastic, with suitable dimensions for covering the face of the patient, the support being cross-hatched with same-size adjacent square marked by borders. The use of the device for evaluating the distribution of acne scars on the face of a patient is also described.

### FIELD OF THE INVENTION

The present invention concerns a method to assess the severity of atrophic acne scars in a patient. It also concerns a device to implement the method of the invention, the device being more particularly used to evaluate the distribution of acne scars.

### TECHNOLOGICAL BACKGROUND OF THE INVENTION

Acne is a frequent skin disorder affecting most individuals at some time in their lives, most often during adolescence (85% of adolescents are confronted with this problem as shown in the study by Mancini A J, Baldwin H E, Eichenfield L F, Friedlander S F, Yan A C. Acne life cycle: the spectrum of pediatric disease. Semin Cutan Med Surg. 2011 September; 30(3 Suppl):S2-5), but also at adult age. Acne most often occurs on the face but can also erupt on the back, chest, shoulders and neck.

A large proportion of persons suffering from acne also develop scars of greater or lesser severity. The true incidence of acne scars is difficult to evaluate, nonetheless recent studies such as the one by Tan & al, (Development and Validation of a Scale for Acne Scar Severity (SCAR-S) of the face and the trunk, 2010 J of cutaneous medicine and surgery vol14, 2010, 156-160) show that 87% of acne sufferers develop acne scars on the face.

Therefore acne scars, and more particularly those on the face, are one of the major concerns of any acne patient, and in particular for adolescents or young adults due to the obvious impact on self-esteem.

Such scars resulting from acne lesions are conventionally described under 3 types, namely erythematous, pigmented scars, hypertrophic scars substantiated by excess tissue, and atrophic scars characterized by loss of tissue.

The mechanisms at the origin of these types of acne scars and the approaches for treatment being very different, the Applicant has focused more particularly on the examination of atrophic acne scars since they are more frequent and represent a majority of the patients' demand.

Different classification systems of acne scars and in particular of atrophic scars have been proposed in the literature.

To facilitate the identification of one same lesion, atrophic acne scars have been intuitively classified according to shape:

“V-shaped” or “ice-pick” shape, having thin, vertical, deep holes;

“U-shaped” or quadrangular (“boxcar”) having depressions with steep, defined edges and regular flat bottom; and

“M-shaped” or “rolling” having broad depressions with gentle sloping edges.

Atrophic acne scars have also been classified according to size, which is usually a secondary characteristic after shape. To that effect, “Ice pick” scars have been defined as being <2 mm according to Jacob et al. (Acne scarring: a classification system and review of treatment options, Journal of the American Academy of Dermatology, 2001, 45:109-117) and Dreno et al. (ECCA grading scale: an original validated acne scar grading scale for clinical practice in dermatology, Dermatology, 2007, 214:46-51) but <1.5 mm in a presentation by Pr. Kang: Communication on 2010 Society for Investigative Disease meeting (May 5-8) in Atlanta, Ga.; quadrangular scars range from 1-4 mm according to Jacob et al. or 2-4 mm according to Dreno et al., and rolling scars can be larger than 4-5 mm.

Different classification systems therefore exist on which scales are based to assess the severity of acne scars.

They concern the evaluation of atrophic acne scars but also hypertrophic, erythematous and pigmented, on the face and even the chest and back, and are used to evaluate the number, size, type and/or colour of these scars.

The publication by Fabbrocini et al. is more particularly known (The management of atrophic acne scars: overview and new tools, J Clin Exp Dermatol Res 2012, S:5, http://dx.doi.org/10.4172/2155-9554.S5-001) which discloses qualitative and quantitative evaluation systems of acne scars.

More particularly, it discloses a first qualitative scale according to Goodman and Baron defined in accordance with the type of scars observed (Postacne scarring: a qualitative global scarring grading system, Dermatologic surgery, vol. 32, no. 12, 1458-1466, 2006). Four different grades from 1 to 4 according to determined degree of severity are provided to evaluate the severity of acne scarring.

However, the notion of scar classification based solely on the shape thereof has poor reproducibility in the setting of a clinical examination. This is in agreement with the preliminary results presented by Finlay et al. (Classification of acne scars is difficult even for acne experts, Journal of European Academy of Dermatology and Venereology: JEADV 2012) showing lack of agreement on the terminology used to describe specific lesions.

Therefore, with this qualitative scale which takes into consideration both atrophic and hypertrophic acne scars as well as erythematous and pigmented acne scars, it is difficult to make a distinction between the different profiles which can be seen in patients.

In addition, this qualitative scale does not take into account the number of scars observed or the notion of scar distribution on the face to determine the severity of acne scars.

Goodman and Baron also developed a second quantitative classification scale which takes into account the number of scars in addition to their qualitative appearance (type of scar) to obtain a score and to determine the severity of acne scars (Postacne scarring—a quantitative global scarring grading system, Journal of Cosmetic Dermatology, vol. 5, no. 1, 48-52, 2006).

However, such a quantitative classification which takes into consideration the number of atrophic and hypertrophic acne scars and also erythematous and pigmented acne scars, does not allow a good distinction to be made between the different categories of atrophic acne scars as per their size and shape and does not take into account either the distribution of acne scars on the face to evaluate acne severity.

The publication by Fabbrocini et al. also discloses a semi-quantitative scale proposed by Dreno et al. (ECCA grading scale: an original validated acne scar grading scale for clinical practice in dermatology, Dermatology, vol. 214, no. 1, 46-51, 2006), namely the scale known as ECCA.

Similar to the aforementioned second scale by Goodman and Baron, such an ECCA scale gives consideration to different types of scars observed according to their size and shape, and to the number thereof and establishes a scoring system to characterize a scale of severity of acne scars.

However, the difficult diagnosis of smaller scars of <2 mm and the presence of numerous scars in one same region leads to counting incoherency and gives rise to insufficient reproducibility among clinicians.

In addition, said classification does not take into account the distribution of acne scars on the face to evaluate the severity of acne scarring.

Finally, Finlay et al. reported the results of a study (Classification of acne scars is difficult even for acne experts, Journal of the European Academy of Dermatology and Venereology: JEADV 2012) based on the internet using high resolution digital images and with open questions on acne scars which were answered by 23 acne experts. In this study, atrophic acne scars were identified very differently by each one, with a mean of five different terms used to identify atrophic acne scars. Those who answered were also questioned on their satisfaction with existing classifications of acne scars, and whereas 61% are unsatisfied, the majority of those who answered are of the opinion that the classification of acne scars affects the choice of suitable treatment.

These results evidence the low consistency between clinical examinations and indicate an obvious need for a standardised classification scheme of acne scars to evaluate the severity thereof.

Additionally, there is very little information in the literature on evaluation by patients themselves of the severity of their acne scars in relation to their number, size and even scar distribution.

For this purpose the SCAR-S scale is more particularly known which assesses the severity of atrophic and hypertrophic acne scars in each region of the face, chest and back and gives a global score (sum of the scores obtained for each of the 3 regions).

According to the clinical trial conducted using the SCAR-S evaluation scale on the face and all 3 regions (Development and validation of a Scale for Acne Scar Severity (SCAR-S) of the face and trunk, Tan J K et al., J Cutan Med Surg. 2010 July-August; 14(4:156-60), the evaluation made by the patient was significantly associated with the scores obtained during the clinical examination made by an expert. The evaluation by patients was based on the question: “Have you got acne scars?” with the possible replies of “none, few, moderate and severe”.

However even if this scale takes into account the evaluation of acne scar severity by patients, it does not appear to take into account how the patient feels with respect to this question asked but is solely based on an observation that is desired to be objective.

As a result, the onset of atrophic acne scar remains an ill-understood phenomenon and the treatment of atrophic acne scars is complex and most often requires the association of several techniques.

The treatment offered is always personalised for each patient whenever possible and generally only concerns the face on account of the cost of techniques which for the most part are not taken in charge by health insurance bodies. In addition, patient information is of essential importance to determine patient motivation and understanding of the expected level of result.

It is therefore important to ensure coherency between dermatologists and other experts with respect to the description and classification of acne scars, in particular atrophic acne scars.

### SUMMARY OF THE INVENTION

Having regard to the foregoing, one problem which the invention sets out to solve is to develop a new scale to evaluate the severity of atrophic acne scars which is easy to apply by clinicians, gives similar results from one clinician to another and by one same clinician between 2 consultations of one same patient (reproducible) so as to obtain clinical significance and which allows best determination of the effect of the best adapted treatment to reduce atrophic acne scars in a given patient.

The Applicant has therefore developed a normalised method to assess the severity of atrophic acne scars in a patient so as to define a systematic approach to guide and improve the clinical evaluation of atrophic acne scars and to standardise discussions between clinicians on suitable treatments to be adopted in accordance with the severity of the cases concerned.

In addition, this normalised method will aid clinicians in evaluating the benefit of a treatment in their patients and in controlling the results of clinical trials conducted on molecules of interest.

The Applicant has also developed and validated a specific tool that will help clinicians apply the method of the invention with good repeatability and reproducibility.

A first subject of the invention to solve this problem is therefore a method to evaluate the severity of atrophic acne scars in a patient, comprising the following steps in which:

at a first qualitative step, the severity of atrophic acne scars is evaluated on the patient's face;

at a second quantitative step which takes into account six different anatomic regions (forehead, left temple, right temple, left cheek, right cheek, jaw but excluding the nose) the atrophic acne scars for each specifically defined region are counted according to size atrophic scars of >4 mm on one hand, and atrophic acne scars of 2-4 mm on the other hand; and

at a third quantitative step, for each of the six specifically defined regions defined at step 2, the distribution of atrophic acne scars is evaluated taking into consideration the scars of <2 mm, 2-4 mm and >4 mm to determine the percentage surface area affected by atrophic scars.

A second subject of the invention is a device to implement the method of the invention, which is in the form of a transparent, flexible, plastic support having dimensions able to cover the patient's face and its different anatomic regions described in FIG. 1 (see below), the support being marked out in adjacent squares of identical size delimited by edging.

Finally, a third subject of the invention is the use of the device of the invention to evaluate the distribution of acne scars on a patient's face.

### BRIEF DESCRIPTION OF THE FIGURES

Other objectives, advantages and characteristics of the invention will be better understood on reading the following description and non-limiting embodiments given with reference to the appended Figures in which:

FIG. 1 shows 3 different views of a face: 1 profile and 2 front views illustrating the six specific anatomic regions defined on a patient's face using the method of the invention;

FIG. 2 schematically illustrates the device of the invention.

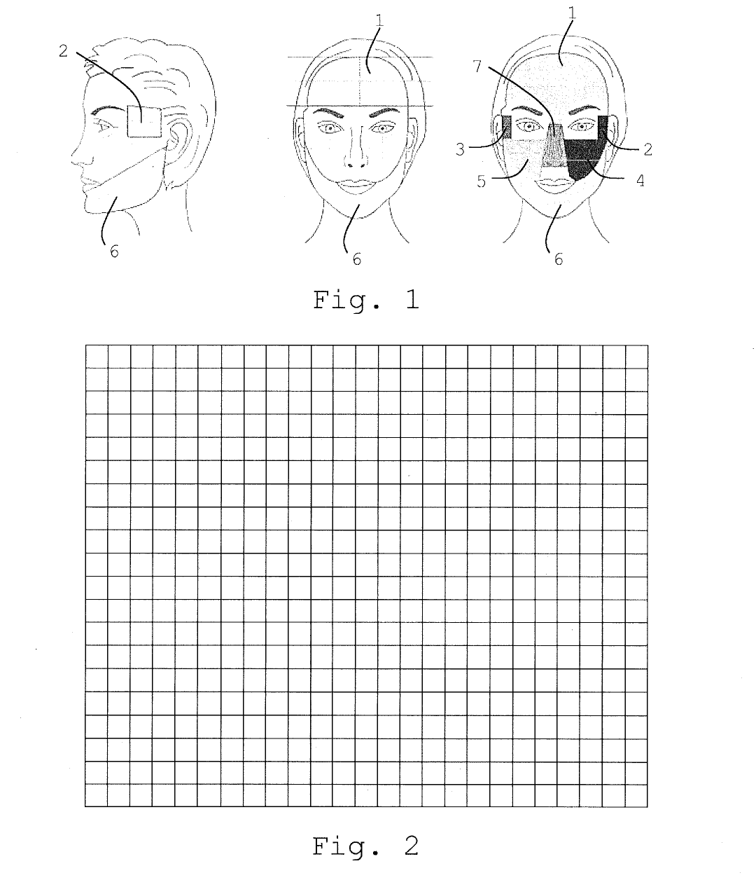

### DETAILED DESCRIPTION OF THE INVENTION

The invention concerns a normalised evaluation method (called “global evaluation of scarring”) to evaluate the severity of atrophic acne scars in a patient.

By “normalised” is meant conditions allowing good repeatability of the evaluation, in particular following precise conditions such as the distance between the patient and the clinician and the use of an adjustable and/or mobile light source.

Advantageously, the clinician must be positioned at a distance of between 20 and 50 cm from the patient to conduct observations.

Those patients able to benefit from the method of the invention are those having atrophic acne scars and possibly suffering from active acne.

The size of the scars according to the invention, in particular atrophic acne scars, represents the maximum length of the scar on the surface of the skin. In the method of the invention, consideration is given for example to atrophic acne scars having a length of less than 2 mm, of between 2-4 mm or longer than 4 mm.

The method of the invention further comprises the following steps of:

at a first qualitative step, evaluating the severity of atrophic acne scars on a patient's face;

at a second quantitative step, in each of the six specifically defined anatomic regions, counting the atrophic acne scars of >4 mm, and the atrophic acne scars of 2-4 mm; and

at a third quantitative step, in each of the six specifically defined anatomic regions defined at step 2, evaluating the distribution of atrophic acne scars on the patient's face taking into account the atrophic scars of <2 mm, 2-4 mm and >4 mm.

This method may further comprise the illumination of the anatomic region concerned using an adjustable and/or mobile light source.

The counting of the atrophic acne scars is performed at a patient-clinician distance of between 20 and 50 cm.

Counts are recorded in a score table of the following type which can be used by the practitioner to determine the percentage of affected surface area, lesion count and dispersion of lesions.

Consultation No 1—Proportion Affected by Scars (the Practitioner Must Use the Grid for this Estimation)

### Forehead L Temple R Temple L Cheek R Cheek Jaw

Consultation No 1—Scar Count (Scars 2 to 4 mm)—to be Entered by the Practitioner

Front L Temple R Temple L Cheek R Cheek Jaw

Consultation no 1—Scar Count (Scars>4 mm)—to be Entered by the Practitioner

Forehead L Temple R Temple L Cheek R Cheek Jaw

The emphasis being on atrophic scars, the consensus is to fine-tune the definition of an atrophic acne scar.

According to the invention, an atrophic acne scar is defined as permanent tissue loss subsequent to an acne lesion, having a diameter larger than that of a normal skin pore. Lesions which could be characterized by post-inflammatory erythema, post-inflammatory hyperpigmentation, scars that are a sequel to chicken-pox and perifollicular elastolysis have been excluded. Similarly, hypertrophic acne scars that are the result of excess tissue are also excluded.

At the first step of the method, evaluation is a global evaluation of atrophic acne scars on the entire face of a patient. Evaluation at the first step of the method advantageously takes in consideration the number and the global distribution on the face of all atrophic acne scars, namely “U-shaped” or “quadrangular” of 2-4 mm, “M-shaped” or “rolling” of >4 mm and also “V-shaped” or “ice pick” scars of <2 mm.

Therefore to increase the repeatability of evaluation with good consistency, and considering the difficult identification of small “ice pick” scars of <2 mm which may be confused with dilated pores, a threshold of < or >4 mm was preferably determined for evaluation at the first step for reliable, reproducible identification during clinical examination.

For this purpose, the evaluation at the first step of the method is advantageously determined using a scale of 0 to 4, with:

0 representing a face not displaying any visible atrophic acne scar;

1 representing a face having a few small atrophic acne scars of <4 mm visible at a distance of 20 to 50 cm dispersed over less 25% of the face;

2 representing a face having some small atrophic acne scars of <4 mm visible at a distance of 20 to 50 cm dispersed over less than 50% of the face;

3 representing a face having many small atrophic acne scars of <4 mm and no more than 3 large atrophic acne scars of >4 mm visible at a distance of 20 to 50 cm dispersed over up to 75% of the face; and

4 representing a face having multiple small atrophic acne scars of <4 mm and large atrophic acne scars of >4 mm visible at a distance of 20 to 50 cm dispersed over more than 75% of the face.

At the second step of the method, for each of the six specifically defined regions, preferably atrophic “ice pick” acne scars of <2 mm are not counted.

In addition, at the second step of the method, six specific anatomic regions of the face are defined, namely: forehead 1, left temple 2, right temple 3, left cheek 4, right cheek 5 and/or jaw 6, (the nose 7 not being considered). For example, the jaw is defined as extending from the corner of the mouth as far as underneath the ear opening.

The counting at the second step for each specifically defined region is advantageously determined according to a classification in a scoring scale.

Advantageously, at step 3 of the method, a device is applied to the patient's face which will help clinicians, using an adjustable and/or mobile light source, to implement the invention and to evaluate the distribution of atrophic acne scars with good repeatability and reproducibility.

By adjustable and/or mobile light source is meant any device able to be operated by a clinician to adjust the angle and luminosity of light on the body surface to be investigated.

As a non-limiting example mention can be made of torches, LED lamps, lamps, fluorescent or halogen examination lamps and surgical lighting devices.

The device is applied to and held on the patient's face when the method is implemented by the patient, by the clinician or by any other person or means allowing the application and holding in position of the device on the patient's face during the clinical examination.

The device such as illustrated in FIG. 2 is in the form of a transparent, flexible plastic support having dimensions able to cover the patient's face, the support being marked out in adjacent squares of identical size delimited by edging.

Advantageously, to meet a practical need for the user and to avoid bulkiness when in use or stored, the dimensions of the device are preferably between 15 and 20 cm in width and between 20 and 30 cm in length e.g. 18 cm×24 cm.

The device is flexible i.e. it can easily be folded without breaking under the pressure applied by the user and can follow the contour of the patient's face when applied.

In addition, the device is transparent so that the user is able to see and count the acne scars through the device, for example at a distance of 20-50 cm, advantageously with the aid of tangential light.

The marking in squares of identical size is advantageous compared with diamond or rectangle shapes, is easy to obtain and offers good repeatability and reproducibility to implement the method of the invention for an objective evaluation from one consultation to another for one same patient and between patients.

The device is preferably marked out in adjacent squares of identical size with sides of between 1 and 2 cm, more preferably sides of 1.5 cm.

The device of the invention is more particularly used for the third step of the method wherein the distribution of acne scarring on the patient's face is evaluated, advantageously at specific regions of the face from among the forehead, left temple, right temple, left cheek, right cheek and/or jaw, the nose not being taken into consideration.

Preferably, the device is used to evaluate the distribution of atrophic acne scars on a patient's face.

Finally at the third step of the method, for each region specifically defined at step 2, the device is used to evaluate the distribution of atrophic acne scars including “ice pick” atrophic acne scars.

For this purpose, the evaluation of the distribution of atrophic acne scars according to the third step, for each specifically defined region, is carried out following the steps below:

the device is suitably placed on the selected specific region and the number of squares covering this region is counted;

the number of squares are counted which comprise at least one atrophic acne scar of 2-4 mm or >4 mm, and grouped atrophic acne scars of <2 mm, and if one same scar is contained in at least 2 squares consideration is solely given to the square in which it is mostly present, unless the 2 or more squares are mostly occupied by such a scar; and

the distribution of acne scars is evaluated for each region as per the ratio:

Number of squares comprising at least one scar in a defined region*100/Number of squares covering the selected region.

The evaluation of the distribution of atrophic acne scars at the third step for each specifically defined region is advantageously determined according to a classification in a graded scale which will allow correlating the evolution of the distributions with a score.

Further advantageously, a global scoring system significantly associates the results obtained at each of the 3, even 4, steps of the method of the invention to determine the best adapted treatment for reducing atrophic acne scars in a given patent and to objectify treatment, for example by choosing the best adapted treatment as a function of the score obtained, for example from among chemical peeling, glycolic acid, Jessner solution (salicylic acid, resorcinol and lactic acid in 95% ethanol), pyruvic acid, salicylic acid, trichloroacetic acid, TCA CROSS (Trichloroacetic Acid Chemical Reconstruction Of Skin Scars), dermabrasion/microdermabrasion, laser treatment, perforation techniques, skin graft, fat filler, injectable implant, drainage, combined therapy.
A cross-reference table between the global score obtained and the effect of treatment between two consultations with the practitioner is advantageously drawn up.
The scoring system defined to evaluate the severity of atrophic acne scars on a patient's face advantageously takes into account the distribution of atrophic acne scars in each region and further advantageously the surface area represented by each face region, namely for example 30% for the forehead, 5% for the left temple, 5% for the right temple, 15% for the left cheek, 15% for the right cheek and 30% for the jaw, the nose not being taken into consideration.

Other advantages and characteristics of the invention will become better apparent on reading the results given below of the study conducted.

The study set out to evidence the repeatability and reproducibility of the evaluation of the severity of atrophic acne scars between different experts during clinical examination carried out on one same patient, applying the method of the invention, and for one same expert between 2 consultations for one same patient.

The study was conducted by 5 dermatology experts in 31 patients such as shown in the Table below, 10 of these patients suffering from active acne, 1 from slight acne and 9 from moderate acne.

| | | |
|---|---|---|
| Gender | Female | 14 (45.2%) |
| | Male | 17 (54.8%) |
| | Total | 31 (100%) |
| Age | n | 31 |
| | Mean | 30.1 |
| | Median | 26 |
| | Standard deviation | 10.8 |
| | (Min, Max) | (18.58) |
| Ethnic origin | Asian - Chinese | 2 (6.5%) |
| | Asian - Other | 1 (3.2%) |
| | Caucasian | 23 (74.2%) |
| | Mixed race | 1 (3.2%) |
| | Negroid/Black | 3 (9.7%) |
| | Other | 1 (3.2%) |
| | Total | 31 (100%) |
| Fitzpatrick | II | 9 (29%) |
| skin types | III | 16 (51.6%) |
| | IV | 2 (6.5%) |
| | V | 2 (6.5%) |
| | VI | 2 (6.5%) |
| | Total | 31 (100%) |

The 2 consultations by one same patient were made on 2 consecutive days.

Each expert applied the 3 first steps of the method of the invention. In addition, each expert used the device of the invention to determine specific face regions to be examined, namely the forehead, left temple, right temple, left cheek, right cheek and jaw and more particularly to implement steps 2 and 3 of the method of the invention.

A coefficient of correlation (ICC) was calculated for each of the steps of the method of the invention, for the evaluation conducted between the experts of one same patient at the same consultation and for one same expert between 2 consultations of one same patient.

The coefficients of correlation are interpreted as follows:

0-0.29 indicates low correlation;
0.30-0.49 indicates fairly good correlation,
0.50-0.69 indicates moderate correlation;
0.70-0.80 indicates strong correlation; and
>0.8 indicates almost perfect correlation.

For the implementation of step 1 of the method of the invention concerning the global evaluation made by each expert, the following correlations were obtained between each expert for each of the 2 consultations and for one same expert over the 2 consultations:

### Consultation 1

| | ICC | Lower limit | Upper limit |
|---|---|---|---|
| Expert 1 and | 0.71 | 0.48709 | 0.84606 |
| Expert 2 | | | |
| Expert 1 and | 0.72 | 0.49333 | 0.8551 |
| Expert 3 | | | |
| Expert 1 and | 0.79 | 0.611 | 0.89215 |
| Expert 4 | | | |
| Expert 1 and | 0.7 | 0.47178 | 0.84032 |
| Expert 5 | | | |
| Expert 2 and | 0.65 | 0.38686 | 0.81525 |
| Expert 3 | | | |
| Expert 2 and | 0.74 | 0.52958 | 0.86464 |
| Expert 4 | | | |
| Expert 2 and | 0.73 | 0.51813 | 0.85746 |
| Expert 5 | | | |
| Expert 3 and | 0.65 | 0.38121 | 0.81746 |
| Expert 4 | | | |
| Expert 3 and | 0.65 | 0.38686 | 0.81525 |
| Expert 5 | | | |
| Expert 4 and | 0.78 | 0.59441 | 0.8867 |
| Expert 5 | | | |

Mean ICC 0.712 (strong correlation)

### Consultation 2

| | ICC | Lower limit | Upper limit |
|---|---|---|---|
| Expert 1 and | 0.69 | 0.45186 | 0.83634 |
| Expert 2 | | | |
| Expert 1 and | 0.85 | 0.71397 | 0.9242 |
| Expert 3 | | | |
| Expert 1 and | 0.72 | 0.49806 | 0.85341 |
| Expert 4 | | | |
| Expert 1 and | 0.62 | 0.33743 | 0.80008 |
| Expert 5 | | | |
| Expert 2 and | 0.72 | 0.50254 | 0.85178 |
| Expert 3 | | | |
| Expert 2 and | 0.76 | 0.56576 | 0.87433 |
| Expert 4 | | | |
| Expert 2 and | 0.72 | 0.49333 | 0.8551 |
| Expert 5 | | | |
| Expert 3 and | 0.81 | 0.64814 | 0.90184 |
| Expert 4 | | | |
| Expert 3 and | 0.62 | 0.34328 | 0.79768 |
| Expert 5 | | | |
| Expert 4 and | 0.73 | 0.50911 | 0.86067 |
| Expert 5 | | | |

| Mean ICC 0.724 (strong correlation) | | | |
|---|---|---|---|
| | ICC | | |
| | intra Expert | Lower limit | Upper limit |
| Expert 1 and | 0.91 | 0.82318 | 0.95524 |
| Expert 1 | | | |
| Expert 2 and | 0.76 | 0.56576 | 0.87433 |
| Expert 2 | | | |
| Expert 3 and | 0.67 | 0.41658 | 0.8268 |
| Expert 3 | | | |
| Expert 4 and | 0.87 | 0.74965 | 0.93465 |
| Expert 4 | | | |
| Expert 5 and | 0.6 | 0.31488 | 0.7858 |
| Expert 5 | | | |

Mean ICC 0.762 (strong correlation)

The results obtained show a strong correlation between each of the experts (0.712 and 0.724) and for one same expert (0.762).

The evaluation at step 1 of the method of the invention is therefore reproducible with strong consistency (0.76) for one same expert and from one expert to another (0.72).

For the implementation of step 2 of the method, and more particularly for the count of atrophic “U-shaped” or quadrangular scars of 2-4 mm, the following coefficients of correlation were obtained between each expert for each of the 2 consultations and for one same expert over 2 consultations:

### Consultation 1

| | ICC | Lower limit | Upper limit |
|---|---|---|---|
| Expert 1 and | 0.51 | 0.20484 | 0.7248 |
| Expert 2 | | | |
| Expert 1 and | 0.82 | 0.66508 | 0.90725 |
| Expert 3 | | | |
| Expert 1 and | 0.86 | 0.73172 | 0.92944 |
| Expert 4 | | | |
| Expert 1 and | 0.64 | 0.38271 | 0.80516 |
| Expert 5 | | | |
| Expert 2 and | 0.64 | 0.38271 | 0.80516 |
| Expert 3 | | | |
| Expert 2 and | 0.29 | −0.06227 | 0.57802 |
| Expert 4 | | | |
| Expert 2 and | 0.18 | −0.17126 | 0.49066 |
| Expert 5 | | | |
| Expert 3 and | 0.69 | 0.45186 | 0.83634 |
| Expert 4 | | | |
| Expert 3 and | 0.48 | 0.16647 | 0.70538 |
| Expert 5 | | | |
| Expert 4 and | 0.7 | 0.46712 | 0.84206 |
| Expert 5 | | | |

Mean ICC 0.581 (moderate correlation)

### Consultation 2

| | ICC | Lower limit | Upper limit |
|---|---|---|---|
| Expert 1 and | 0.67 | 0.42176 | 0.8248 |
| Expert 2 | | | |
| Expert 1 and | 0.65 | 0.39219 | 0.81314 |
| Expert 3 | | | |
| Expert 1 and | 0.78 | 0.59825 | 0.88542 |
| Expert 4 | | | |
| Expert 1 and | 0.42 | 0.086558 | 0.66882 |
| Expert 5 | | | |
| Expert 2 and | 0.87 | 0.75226 | 0.9339 |
| Expert 3 | | | |
| Expert 2 and | 0.38 | 0.045081 | 0.63813 |
| Expert 4 | | | |
| Expert 2 and | 0.12 | −0.23017 | 0.44266 |
| Expert 5 | | | |
| Expert 3 and | 0.32 | −0.023296 | 0.59579 |
| Expert 4 | | | |
| Expert 3 and | 0.02 | −0.32296 | 0.35831 |
| Expert 5 | | | |
| Expert 4 and | 0.7 | 0.47178 | 0.84032 |
| Expert 5 | | | |

| Mean ICC 0.493 (fairly good correlation) | | | |
|---|---|---|---|
| | ICC | | |
| | intra Expert | Lower limit | Upper limit |
| Expert 1 and | 0.75 | 0.54556 | 0.87019 |
| Expert 1 | | | |
| Expert 2 and | 0.81 | 0.64814 | 0.90184 |
| Expert 2 | | | |
| Expert 3 and | 0.6 | 0.32587 | 0.78106 |
| Expert 3 | | | |
| Expert 4 and | 0.75 | 0.054556 | 0.87019 |
| Expert 4 | | | |
| Expert 5 and | 0.78 | 0.59825 | 0.88542 |
| Expert 5 | | | |

Mean ICC 0.738 (strong correlation)

The results obtained show fairly good to moderate correlation between each of the experts (0.493 and 0.581) and strong correlation for one same expert (0.738).

The counting of atrophic acne scars of 2-4 mm such as defined at step 2 of the method of the invention is therefore reproducible with strong consistency (0.74) for one same expert and with moderate correlation (0.54) from one expert to another.

For the implementation of step 2 and more particularly for the counting of “M-shaped” or rolling atrophic acne scars of >4 mm, the following coefficients of correlation were obtained:

### Consultation 1

| | ICC | Lower limit | Upper limit |
|---|---|---|---|
| Expert 1 and | 0.74 | 0.52958 | 0.86463 |
| Expert 2 | | | |
| Expert 1 and | 0.77 | 0.58193 | 0.87989 |
| Expert 3 | | | |
| Expert 1 and | 0.59 | 0.30656 | 0.77733 |
| Expert 4 | | | |
| Expert 1 and | 0.79 | 0.61473 | 0.89092 |
| Expert 5 | | | |
| Expert 2 and | 0.63 | 0.36314 | 0.80134 |
| Expert 3 | | | |
| Expert 2 and | 0.46 | 0.12938 | 0.69857 |
| Expert 4 | | | |
| Expert 2 and | 0.56 | 0.26541 | 0.75896 |
| Expert 5 | | | |
| Expert 3 and | 0.39 | 0.050839 | 0.64851 |
| Expert 4 | | | |
| Expert 3 and | 0.73 | 0.51813 | 0.85746 |
| Expert 5 | | | |
| Expert 4 and | 0.4 | 0.06265 | 0.65532 |
| Expert 5 | | | |

Mean ICC 0.606 (moderate correlation)

### Consultation 2

| | ICC | Lower limit | Upper limit |
|---|---|---|---|
| Expert 1 and | 0.93 | 0.86107 | 0.96537 |
| Expert 2 | | | |
| Expert 1 and | 0.66 | 0.40691 | 0.81899 |
| Expert 3 | | | |
| Expert 1 and | 0.73 | 0.51813 | 0.85746 |
| Expert 4 | | | |
| Expert 1 and | 0.79 | 0.611 | 0.89215 |
| Expert 5 | | | |
| Expert 2 and | 0.73 | 0.51813 | 0.85746 |
| Expert 3 | | | |
| Expert 2 and | 0.61 | 0.33989 | 0.78714 |
| Expert 4 | | | |
| Expert 2 and | 0.85 | 0.71688 | 0.92332 |
| Expert 5 | | | |
| Expert 3 and | 0.2 | −0.15105 | 0.50625 |
| Expert 4 | | | |
| Expert 3 and | 0.9 | 0.80662 | 0.94955 |
| Expert 5 | | | |
| Expert 4 and | 0.37 | 0.033463 | 0.63118 |
| Expert 5 | | | |

| Mean ICC 0.677 (moderate correlation) | | | |
|---|---|---|---|
| | ICC | | |
| | intra Expert | Lower limit | Upper limit |
| Expert 1 and | 0.89 | 0.78605 | 0.945 |
| Expert 1 | | | |
| Expert 2 and | 0.78 | 0.59441 | 0.8867 |
| Expert 2 | | | |
| Expert 3 and | 0.82 | 0.66508 | 0.90725 |
| Expert 3 | | | |
| Expert 4 and | 0.91 | 0.82318 | 0.95524 |
| Expert 4 | | | |
| Expert 5 and | 0.76 | 0.56576 | 0.87433 |
| Expert 5 | | | |

Mean ICC 0.832 (almost perfect correlation)

The results obtained show a moderate correlation between each of the experts (0.606 and 0.677) and almost perfect correlation for one same expert (0.832).

The counting of atrophic acne scars of >4 mm such as defined at step 2 of the method of the invention is therefore reproducible with almost perfect consistency (0.83) for one same expert and with moderate correlation (0.64) from one expert to another.

For the implementation of step 3 of the method of the invention to evaluate the distribution of atrophic acne scars performed by each expert, the following coefficients of correlation were obtained between each expert for each of the 2 consultations, and for one same expert over the 2 consultations:

### Consultation 1

| | ICC | Lower limit | Upper limit |
|---|---|---|---|
| Expert 1 and | — | — | — |
| Expert 2 | | | |
| Expert 1 and | 0.91 | 0.8251 | 0.95471 |
| Expert 3 | | | |
| Expert 1 and | 0.82 | 0.66174 | 0.9083 |
| Expert 4 | | | |
| Expert 1 and | 0.76 | 0.56576 | 0.87433 |
| Expert 5 | | | |
| Expert 2 and | — | — | — |
| Expert 3 | | | |
| Expert 2 and | — | — | — |
| Expert 4 | | | |
| Expert 2 and | — | — | — |
| Expert 5 | | | |
| Expert 3 and | 0.8 | 0.62775 | 0.89756 |
| Expert 4 | | | |
| Expert 3 and | 0.63 | 0.36831 | 0.79919 |
| Expert 5 | | | |
| Expert 4 and | 0.63 | 0.36314 | 0.80134 |
| Expert 5 | | | |

Mean ICC 0.758 (strong correlation)

### Consultation 2

| | ICC | Lower limit | Upper limit |
|---|---|---|---|
| Expert 1 and | 0.8 | 0.62775 | 0.89756 |
| Expert 2 | | | |
| Expert 1 and | 0.86 | 0.73172 | 0.92944 |
| Expert 3 | | | |
| Expert 1 and | 0.86 | 0.73172 | 0.92944 |
| Expert 4 | | | |
| Expert 1 and | 0.75 | 0.54556 | 0.87019 |
| Expert 5 | | | |
| Expert 2 and | 0.88 | 0.7702 | 0.93914 |
| Expert 3 | | | |
| Expert 2 and | 0.79 | 0.61473 | 0.89092 |
| Expert 4 | | | |
| Expert 2 and | 0.56 | 0.27095 | 0.75641 |
| Expert 5 | | | |
| Expert 3 and | 0.79 | 0.61473 | 0.89092 |
| Expert 4 | | | |
| Expert 3 and | 0.66 | 0.41187 | 0.81701 |
| Expert 5 | | | |
| Expert 4 and | 0.68 | 0.44156 | 0.82873 |
| Expert 5 | | | |

| Mean ICC 0.763 (strong correlation) | | | |
|---|---|---|---|
| | ICC | | |
| | intra Expert | Lower limit | Upper limit |
| Expert 1 and | 0.93 | 0.86107 | 0.96537 |
| Expert 1 | | | |
| Expert 2 and | — | — | — |
| Expert 2 | | | |
| Expert 3 and | 0.9 | 0.80662 | 0.94955 |
| Expert 3 | | | |
| Expert 4 and | 0.83 | 0.67898 | 0.91363 |
| Expert 4 | | | |
| Expert 5 and | 0.74 | 0.53386 | 0.86311 |
| Expert 5 | | | |

Mean ICC 0.85 (almost perfect correlation)

The results obtained show a strong correlation between each of the experts (0.758 and 0.763) and almost perfect correlation for one same expert (0.85).

The evaluation of the distribution of atrophic acne scars at step 3 of the method of the invention is therefore reproducible with almost perfect consistency (0.85) for one same expert and with strong correlation (0.76) from one expert to another.

To conclude and as illustrated by the results of the study presented above, the implementation of the method of the invention shows reproducibility with moderate to strong consistency from one expert to another, and with a strong to almost perfect consistency for one same expert.

It follows that the reproducibility of the evaluation of the severity of atrophic acne scars for one same expert and above all from one expert to another is therefore substantially improved with the method of the invention compared with the evaluations performed using existing classifications for which reproducibility has low consistency.

In addition, the results obtained with the 10 patients suffering from active acne of slight severity are in conformity with the results obtained for all 31 patients. A patient's active acne does not therefore modify the application of the method of the invention or the reproducibility of evaluation with strong to almost perfect consistency when severity is slight.

With the method of the invention it is therefore possible to evaluate the severity of atrophic acne scars in a patient having clinical significance both for one same expert and from one expert to another.

Of course, the invention is not limited to the described embodiment illustrated in the appended Figures, and it is within the reach of persons skilled in the art having recourse to routine operations to carry out other embodiments that have not been explicitly described without departing from the framework or scope of the present invention.

1. A method of evaluating severity of atrophic acne scars in a patient, the method comprising the following steps:
in a first qualitative step, evaluating the severity of the atrophic acne scars on the patient's face;
in a second quantitative step, for each specifically defined region, counting atrophic acne scars of >4 mm, and counting atrophic acne scars of 2-4 mm; and
in a third quantitative step, for each region specifically defined at step 2, evaluating the distribution of the atrophic acne scars taking into consideration atrophic scars of <2 mm, 2-4 mm and >4 mm.

2. The method as defined by claim 1, wherein the evaluation at the first qualitative step takes into consideration the number and the distribution on the face of atrophic acne scars of <2 mm, 2-4 mm and >4 mm.

3. The method as defined by claim 2, wherein the evaluation at the first qualitative step is determined on a scale of 0 to 4 with:
0 representing a face having no visible atrophic acne scar;
1 representing a face having a few small atrophic acne scars of <4 mm visible at a distance of 20 to 50 cm dispersed over less than 25% of the face;
2 representing a face having some atrophic acne scars of <4 mm visible at a distance of 20 to 50 cm dispersed over less than 50% of the face;
3 representing a face having many small atrophic acne scars of <4 mm and no more than 3 large atrophic acne scars of >4 mm visible at a distance of 20 to 50 cm dispersed over up to 75% of the face; and
4 representing a face having multiple small atrophic acne scars of <4 mm and large atrophic acne scars of >4 mm visible at a distance of 20 to 50 cm dispersed over more than 75% of the face.

4. The method as defined by claim 1, wherein the evaluation of the distribution of acne scars is conducted at specific regions of the face selected from the group consisting of the forehead (1), left temple (2), right temple (3), left cheek (4), right cheek (5) and/or the jaw (6), wherein the nose (7) is not taken into consideration, and wherein for each specifically defined region, the atrophic acne scars of <2 mm are not counted.

5. The method as defined by claim 1, wherein at steps 2 and 3, a device is applied to the patient's face, the device being in the form of a transparent, flexible plastic support having dimensions able to cover the patient's face, the support being marked out in adjacent squares of identical size delimited by edging.

6. The method as defined by claim 5, wherein the support is additionally marked out in squares with different edgings to defined specific regions of the face selected from the group consisting of the forehead (1), left temple (2), right temple (3), left cheek (4), right cheek (5) and/or the jaw (6), and wherein the nose is not taken into consideration.

7. The method as defined by claim 5, wherein at the third step, for each region specifically defined at step 2, the device is used to evaluate the distribution of atrophic acne scars also taking into consideration atrophic acne scars of <2 mm

8. The method as defined by claim 7, wherein the distribution of atrophic acne scars at the third step for each specifically defined region is determined using the following steps:
placing the device on the selected specific region and counting the number of squares covering this region;
counting the number of squares which contain at least one atrophic acne scar of 2-4 mm or >4 mm, atrophic acne scars of <2 mm, and if one same scar is contained in at least 2 squares, counting solely the square in which it is mostly contained, unless the 2 squares or more are mostly occupied by such a scar; and
evaluating the distribution of atrophic acne scars in each region using the ratio: Number of squares containing at least one scar for a defined region*100/Number of squares covering the selected region.

9. A device for implementing the method as defined by claim 1, wherein it is in the form of a transparent, flexible, plastic support having dimensions able to cover the patient's face, the support being marked out in adjacent squares of identical size delimited by edging.

10. The device as defined by claim 9, wherein the support is marked out in adjacent squares having sides of between 1 cm and 2 cm.

11. The device as defined by claim 10, wherein the support is marked out in squares having sides of 1.5 cm.

12. The device as defined by claim 9, wherein the support is additionally marked out in squares having different edgings so as to define specific regions of the face selected from the group consisting of the forehead (1), left temple (2), right temple (3), left cheek (4), right cheek (5) and/or the jaw (6), and wherein the nose is not taken into consideration.

13. The device as defined by claim 9, wherein the dimensions of the support are between 15 cm and 20 cm in width and between 20 cm and 30 cm in length.

14. A method of evaluating distribution of acne scars on a patients face, the method comprising using the device as defined by claim 9, to evaluate the distribution of the acne scars on the patient's face.

15. The method as defined by claim 14, wherein the acne scars evaluated are atrophic acne scars.
